(19) United States
(12) Patent Application Publication
(10) Pub. No.: US 2025/0366931 A1
(43) Pub. Date: Dec. 4, 2025

(54) IMAGE PROCESSING APPARATUS, IMAGE PROCESSING METHOD, AND IMAGE PROCESSING PROGRAM

(71) Applicant: FUJIFILM Corporation, Tokyo (JP)

(72) Inventor: Motoshi MASUDA, Tokyo (JP)

(73) Assignee: FUJIFILM Corporation, Tokyo (JP)

(21) Appl. No.: 19/221,533

(22) Filed: May 29, 2025

(30) Foreign Application Priority Data
Jun. 4, 2024 (JP) 2024-090893

(51) Int. Cl. A61B 34/20; A61B 6/46; A61B 10/02; A61B 90/00

(52) U.S. Cl. CPC A61B 34/20; A61B 6/463; A61B 10/0233; A61B 2034/2065; A61B 2090/3762

(57) ABSTRACT

A controller performs processing of superimposing and displaying on a CT image a virtual position reached by a distal end of an inner sleeve, based on predetermined distance information from an outer sleeve to the distal end of the inner sleeve in a case where the inner sleeve protrudes from the outer sleeve.

### CROSS-REFERENCE TO RELATED APPLICATIONS

This application claims priority under 35 USC 119 from Japanese Patent Application No. 2024-090893 filed on Jun. 4, 2024, the disclosure of which is incorporated by reference herein.

### BACKGROUND

### 1. Technical Field

The present disclosure relates to an image processing apparatus, an image processing method, and an image processing program.

### 2. Related Art

JP2000-217814A proposes a three-dimensional image display device that reconstructs a three-dimensional image of a subject including a needle inserted into the subject using a three-dimensional original image of the subject including the needle and displays the three-dimensional image, the three-dimensional image display device comprises a designation unit that designates the needle on the three-dimensional image; a unit that obtains a current position of the needle based on the designation by the designation unit and calculates an extension line in a major axis direction of the needle; and a unit that displays the calculated extension line of the needle on the three-dimensional image.

JP2012-518453A proposes a system and a method for supporting or executing image-guided transjugular intrahepatic portosystemic shunt placement in a part of a biological structure of a patient, the system including a guide needle part including a hollow tube having a bend toward a most distal end and a portion of a biopsy needle including an element indicating at least one position at the distal end of the needle.

JP2003-325503A proposes an image display device comprising a detection unit that detects an end point of a needle from an X-ray tomographic image including an image of the needle, and a notification unit that specifies a display unit in which the end point of the needle detected by the detection unit is displayed among a plurality of display units and notifies the display unit.

### SUMMARY

In a case of puncturing with a biopsy needle while confirming a radiation image, such as a CT-guided biopsy, the outer sleeve portion of the biopsy needle is inserted first while confirming the position by the radiation, and the manual distance measurement is performed on the radiation image at a point in time when a distal end of the biopsy needle approaches the target. Then, after the distal end of the outer sleeve portion is made to reach a position in consideration of a distance of the inner sleeve protruding from the outer sleeve, the inner sleeve is inserted and protruded from the outer sleeve to collect the tissue.

In the related art, since the manual distance measurement is performed on a radiation image, it is necessary to perform the distance measurement each time the positional relationship between the distal end of the outer sleeve and the target changes, which takes time.

Therefore, an object of the present disclosure is to provide an image processing apparatus, an image processing method, and an image processing program capable of easily confirming a protrusion distance of an inner sleeve of the biopsy needle, compared to a case of performing manual distance measurement.

In order to achieve the above object, according to a first aspect of the present disclosure, there is provided image processing apparatus comprising a processor, in which the processor is configured to acquire a radiation image of a subject including a biopsy needle in which an inner sleeve protrudes from an outer sleeve; detect a biopsy needle region in the radiation image; and based on a result of the detection, superimpose and display on the radiation image a virtual position reached by a distal end of the inner sleeve protruding from the outer sleeve.

A second aspect according to the present disclosure provides the image processing apparatus according to the first aspect, in which the processor is configured to superimpose and display the virtual position on the radiation image based on predetermined distance information to the distal end of the inner sleeve in a case where the inner sleeve protrudes from the outer sleeve.

A third aspect according to the present disclosure provides the image processing apparatus according to the second aspect, in which the processor is configured to, based on the predetermined distance information, calculate a position of the distal end of the inner sleeve in accordance with a display magnification, and superimpose and display the virtual position on the radiation image.

A fourth aspect according to the present disclosure provides the image processing apparatus according to the second aspect, in which the processor is configured to, based on the predetermined distance information, calculate a position of the distal end of the inner sleeve in accordance with a cross section of the radiation image to be displayed, and superimpose and display the virtual position on the radiation image.

A fifth aspect according to the present disclosure provides the image processing apparatus according to the first aspect, in which the processor is configured to perform labeling processing and edge processing on the radiation image; calculate an average radiation density value for each pixel within an object labeled by the labeling processing; and detect, among objects with a density value range equal to or more than a preset threshold value, at least one object having a region that extends radially from an image center by a distance equal to or greater than a predetermined distance or an object having a region that extends by a distance equal to or greater than a predetermined distance in a boundary region between air and a body surface and in an air-side region, and set the detected object as a biopsy needle region.

A sixth aspect according to the present disclosure provides the image processing apparatus according to the fifth aspect, in which, among the objects with the density value range equal to or more than the preset threshold value, in a case of detecting the object having the region that extends radially from the image center by the distance equal to or greater than the predetermined distance or the object having the region that extends by the distance equal to or greater than the predetermined distance in the boundary region between the air and the body surface and in the air-side region, the processor is configured to first detect the object having the region that extends by the distance equal to or greater than the predetermined distance in the boundary region between the air and the body surface and in the air-side region, and set the object as a biopsy needle region in a case where the object is detected.

A seventh aspect according to the present disclosure provides the image processing apparatus according to the sixth aspect, in which the processor is configured to detect the object having the region that extends by the distance equal to or greater than the predetermined distance in the boundary region between the air and the body surface and in the air-side region, and in a case where the object is not detected, detect the object having the region that extends radially from the image center by the distance equal to or greater than the predetermined distance.

A eighth aspect according to the present disclosure provides the image processing apparatus according to the fifth aspect, in which the processor is configured to calculate a central pixel between edges of the biopsy needle region and set the central pixel as a pixel of a central axis of the biopsy needle; and superimpose and display the virtual position on the radiation image in a display aspect of at least one of a predetermined color or a predetermined transparency as an inner sleeve that protrudes by a pixel of a protrusion distance of the inner sleeve in a central axis direction of the biopsy needle from a pixel of the distal end of the biopsy needle.

A ninth aspect according to the present disclosure provides the image processing apparatus according to the eighth aspect, in which the processor is configured to calculate the central pixel by interpolating the pixel in a case where edge information is insufficient at the distal end of the biopsy needle.

A tenth aspect according to the present disclosure provides the image processing apparatus according to the first aspect, in which the processor is configured to acquire designation information on a biopsy needle region on the radiation image, which is designated by a user; fill pixels in a density value range equal to or more than a preset threshold value around the designation information based on the designation information to extract the biopsy needle region; and set, after the region of biopsy needle is extracted, a distal end of a region on a center side of the radiation image as a biopsy needle tip.

An image processing method according to an eleventh aspect of the present disclosure for causing a computer to execute processing, the method comprises acquiring a radiation image of a subject including a biopsy needle in which an inner sleeve protrudes from an outer sleeve; detecting a biopsy needle region in the radiation image; and based on a result of the detection, superimposing and displaying a position of a distal end of the inner sleeve on the radiation image as in a case where the inner sleeve protrudes from the outer sleeve, before the inner sleeve protrudes from the outer sleeve.

An image processing program according to a twelfth aspect of the present disclosure for causing a computer to execute processing of acquiring a radiation image of a subject including a biopsy needle in which an inner sleeve protrudes from an outer sleeve; detecting a biopsy needle region in the radiation image; and based on a result of the detection, superimposing and displaying a position of a distal end of the inner sleeve on the radiation image as in a case where the inner sleeve protrudes from the outer sleeve, before the inner sleeve protrudes from the outer sleeve.

According to the present disclosure, it is possible to provide an image processing apparatus, an image processing method, and an image processing program capable of easily confirming a distance of an inner sleeve that protrudes from a biopsy needle, compared to a case of performing manual distance measurement.

### BRIEF DESCRIPTION OF THE DRAWINGS

Exemplary embodiments of the technology of the disclosure will be described in detail based on the following figures, wherein:

FIG. 1 is a view showing a medical image examination apparatus according to the present embodiment;

FIG. 2 is a block diagram showing a configuration of the medical image examination apparatus according to the present embodiment;

FIG. 3 is a diagram showing an example of a biopsy needle used for a CT-guided biopsy;

FIG. 4 is a diagram showing an example in which a virtual position reached by a distal end of the inner sleeve protruding from the outer sleeve is displayed by being superimposed on a CT image;

FIG. 5 is a diagram for describing pixel interpolation of an edge portion of a distal end portion of the outer sleeve;

FIG. 6 is a diagram showing an example of the virtual position of the inner sleeve displayed on a sagittal cross section;

FIG. 7 is a diagram showing an example of the virtual position of the inner sleeve displayed on an axial cross section;

FIG. 8 is a diagram showing an example of a distance at which the virtual position of the inner sleeve is displayed on an axial cross section;

FIG. 9 is a diagram showing an example of the virtual position of the inner sleeve displayed on a coronal cross section; and

FIG. 10 is a flowchart showing an example of a flow of processing performed by the controller of the medical image examination apparatus according to the present embodiment.

### DETAILED DESCRIPTION

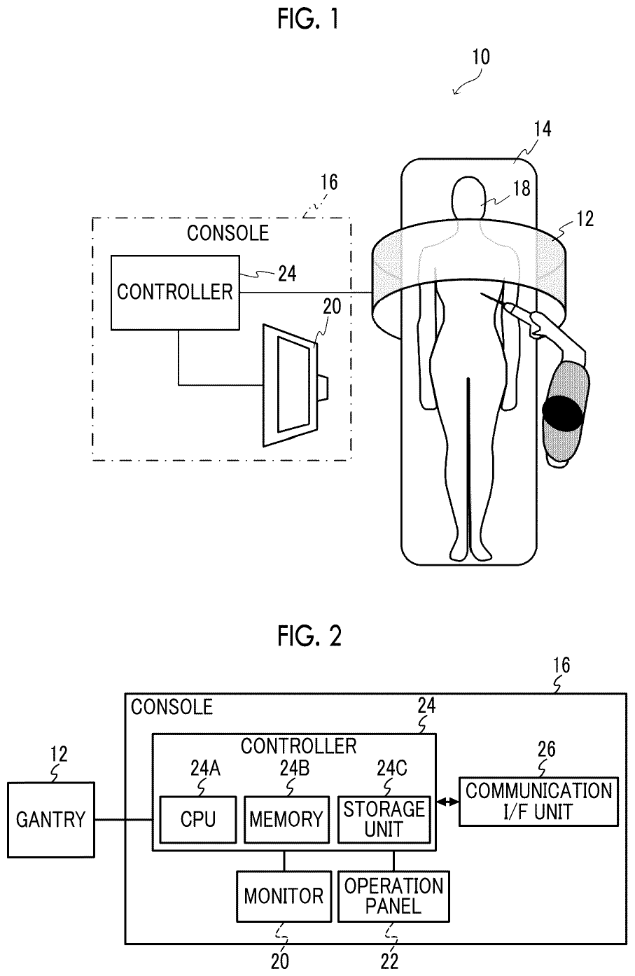

Embodiments of the present invention will be described in detail below with reference to the drawings. The present embodiment does not limit the present invention. FIG. 1 is a view showing a medical image examination apparatus according to the present embodiment, and FIG. 2 is a block diagram showing a configuration of the medical image examination apparatus according to the present embodiment.

The medical image examination apparatus 10 according to the present embodiment comprises a gantry (mounting frame) 12 that acquires a CT image as an example of a radiation image, a bed 14 on which a subject 18, who is an example of the subject 18, lies, and a console 16 that is a computer to be operated.

As is well known, the gantry 12 irradiates the subject 18 lying on the bed 14 with radiation (for example, X-rays) to perform computed tomography (CT) and acquire a CT image. In the CT image, each tomographic image of a body axis cross section, a sagittal cross section, and a coronal cross section is acquired. The gantry 12 is installed in, for example, an imaging room of a radiology department in a medical facility.

The console 16 comprises a monitor 20 that displays the CT image acquired by the gantry 12, and an operation panel 22 that is operated by an operator such as a radiology technician.

As shown in FIG. 2, the gantry 12, the monitor 20, and the operation panel 22 are connected to a controller 24, and the controller 24 comprises a CPU 24A, a memory 24B, and a storage unit 24C as an example of a processor.

The controller 24 also functions as an image processing apparatus that performs image processing on data output from the gantry 12 to generate a tomographic image by loading the image processing program stored in the storage unit 24C into the memory 24B and executing the image processing program by the CPU 24A.

In addition, a communication interface (I/F) unit 26 is connected to the controller 24, and information such as a CT image can be exchanged with an external device.

The console 16 is an example of an “image processing apparatus” according to the technology of the present disclosure. The console 16 also functions as an image display device that displays the generated tomographic image.

The medical image examination apparatus 10 according to the present embodiment is used for CT-guided biopsy in which a skin surface is punctured with a biopsy needle and a part of a tissue is collected while confirming a site of a lesion in, for example, the CT image displayed on the monitor 20.

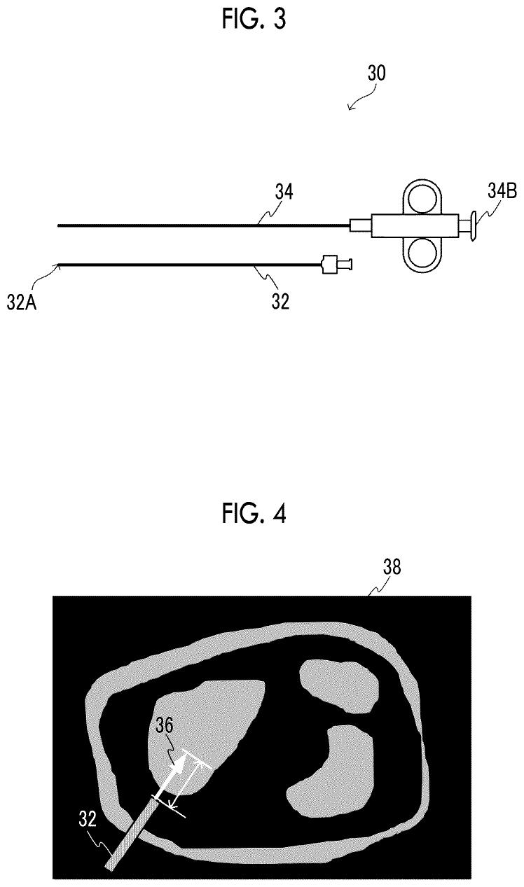

FIG. 3 is a diagram showing an example of a biopsy needle used for a CT-guided biopsy.

As shown in FIG. 3, the biopsy needle 30 comprises an outer sleeve 32 and an inner sleeve 34, and the inner sleeve 34 is inserted into the outer sleeve 32 and protrudes from a distal end 32A of the outer sleeve 32 by pressing a rear end portion 34B of the inner sleeve 34.

Here, an examination procedure of the CT-guided biopsy performed while confirming the CT image displayed on the monitor 20 will be described.

First, a lesion part is imaged by the gantry 12, and the puncture path of the needle is determined in consideration of the distance to the lesion, the angle of needle insertion, whether a thick trachea or blood vessel can be avoided, and the like based on the CT image acquired by the gantry 12.

Under local anesthesia, the outer sleeve 32 of the biopsy needle 30 is inserted, and the outer sleeve 32 of the biopsy needle 30 is cautiously advanced while confirming an advancement direction of the outer sleeve 32 of the biopsy needle 30 by CT imaging.

After it is confirmed that outer sleeve can be punctured up to directly above the target such as the tumor, the inner sleeve 34 is inserted into the outer sleeve 32, and the rear end portion 34B of the inner sleeve 34 is pressed to protrude the inner sleeve 34 from the distal end 32A of the outer sleeve 32 to collect the tissue of the target.

By the way, in the CT-guided biopsy in the related art, the portion of the outer sleeve 32 of the biopsy needle 30 is first inserted while confirming the position in the CT image, and the manual distance measurement is performed on the CT image at a time point when the distal end 32A approaches the target. For example, in the related art, it is necessary to perform distance measurement by performing a manual operation such as designating two points on the screen of the monitor 20. Then, after the distal end 32A of the outer sleeve 32 reaches a position in consideration of the distance of the inner sleeve 34 protruding from the outer sleeve 32, the inner sleeve 34 is inserted to collect the tissue.

As described above, in the CT-guided biopsy in the related art, since the distance measurement of the inner sleeve 34 protruding from the outer sleeve 32 is performed manually, it is necessary to perform the measurement again each time the positional relationship between the distal end 32A of the outer sleeve 32 of the biopsy needle 30 and the target changes, and it takes time to collect the tissue of the target.

In addition, in the case of the distance measurement, it is necessary to consider the position of the distal end 32A of the outer sleeve 32 and whether the outer sleeve 32 and the target are on the same straight line, and it takes time for confirmation in this respect as well.

Therefore, in the medical image examination apparatus 10 according to the present embodiment, the controller 24 acquires the CT image of the subject 18 including the biopsy needle 30, detects a region of the biopsy needle 30 in the CT image, and based on a result of the detection, performs processing of superimposing and displaying on the CT image the virtual position reached by the distal end of the inner sleeve 34 protruding from the outer sleeve 32. For example, as shown in FIG. 4, a virtual position 36 of the inner sleeve 34, which is protruding from the outer sleeve 32, may be displayed and superimposed on the CT image 38. FIG. 4 is a diagram illustrating an example in which the virtual position 36, which is reached by the distal end of the inner sleeve 34 protruding from the outer sleeve 32, is superimposed on and displayed on the CT image 38. In FIG. 4, the virtual position 36 reached by the distal end of the inner sleeve is a distal end of the arrow.

Specifically, the controller 24 performs processing of superimposing and displaying the virtual position 36 reached by the distal end of the inner sleeve 34, on the CT image 38, based on predetermined distance information from the outer sleeve 32 to the distal end of the inner sleeve 34 in a case where the inner sleeve 34 protrudes from the outer sleeve 32. That is, since the distance at which the inner sleeve 34 protrudes from the outer sleeve 32 is determined by the type of the biopsy needle 30 and the like, the virtual position 36 can be specified and displayed from the predetermined distance information for each biopsy needle 30.

More specifically, the controller 24 performs labeling processing and edge extraction processing on the CT image 38 including the biopsy needle 30. An average density value (CT value) at each pixel in the object labeled by the labeling processing is calculated, and an object within a CT value range equal to or greater than a preset threshold value is extracted. Here, the “average density value at each pixel in the object” refers to a value obtained by summing and averaging the density values (for example, CT values) of each pixel constituting the object. The averaged value represents an average of pixel-by-pixel density values and is treated as a representative value of the object. In this context, the term “averaged density” can also be viewed as an index obtained by averaging the CT values (or pixel values) of each pixel included in the object obtained through labeling. By referring not only to the value of a single pixel but also to the “average value on an object-by-object basis,” misdetection can be reduced and the biopsy needle region can be detected with greater accuracy. This approach is considered highly useful for automating or semi-automating the biopsy needle detection process. In order to separate the bone having a CT value close to that of the biopsy needle 30 (metal), only the object corresponding to at least one of the following condition 1 or condition 2 is extracted and treated as the biopsy needle region.
Condition 1. A region that radially from the image center by a distance equal to or greater than a predetermined distance exists.
Condition 2. A region that extends by a distance equal to or greater than a predetermined distance exists in a boundary region between air and a body surface and in an air-side region.

In a case where an object corresponding to each of Condition 1 and Condition 2 is extracted, in a case where the object corresponding to Condition 1 and the object corresponding to Condition 2 are different from each other, the object corresponding to Condition 2 is set as the biopsy needle region. In addition, for Condition 1 and Condition 2, the region corresponding to Condition 2 may be extracted, the region corresponding to Condition 2 may be set as the biopsy needle region in a case where the region corresponding to Condition 2 exists, and in a case the region corresponding to Condition 2 does not exist, the region corresponding to Condition 1 may be extracted, and the region corresponding to Condition 1 may be set as the biopsy needle region in a case where the region corresponding to Condition 1 exists.

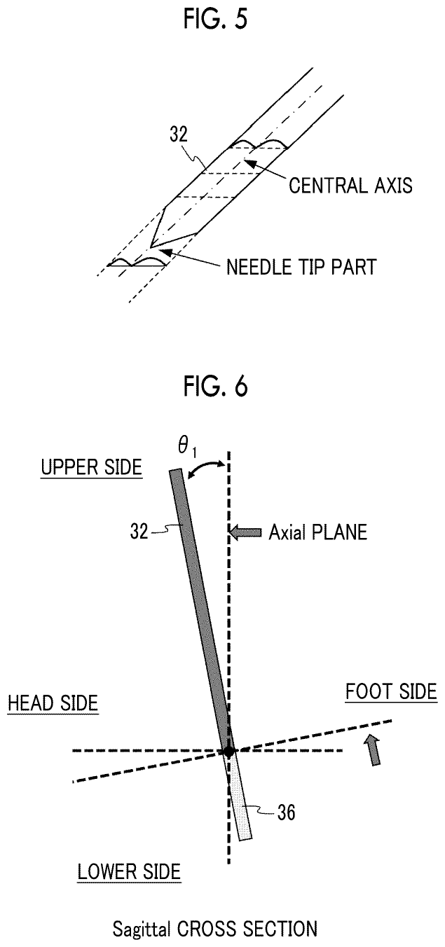

After extracting the biopsy needle region, the distal end of the region on an image center side is set as the biopsy needle tip. A central pixel between the edges of the biopsy needle region is calculated and treated as a pixel of the central axis of the biopsy needle 30. In a case where edge information is insufficient at the needle tip, the pixel is interpolated to calculate the central pixel. For example, in the distal end portion of the outer sleeve 32, as shown by a dotted line in FIG. 5, since the edge information is insufficient and the central pixel cannot be calculated, the central pixel is calculated by interpolating the pixels of the edge portion of the needle up to the needle tip part to derive a central axis pixel. FIG. 5 is a diagram for describing pixel interpolation of an edge portion of a distal end portion of the outer sleeve 32.

In a case of displaying the virtual position 36 reached by the distal end of the inner sleeve 34, the virtual position 36 may be superimposed and displayed on the CT image 38 in at least one display aspect of a predetermined color or transparency as the inner sleeve 34 that protrudes by a pixel of a predetermined protrusion distance of the inner sleeve 34 in a central axis direction of the biopsy needle 30 from a pixel of the biopsy needle tip. For the display aspect, the display is performed using at least one of a color or transparency being distinguishable from the inner sleeve 34 as actually displayed on the CT image 38.

In addition, in a case where display magnification can be changed, the virtual position 36 reached by the distal end of the inner sleeve 34 changes depending on the display magnification. Therefore, the controller 24 may calculate and display the virtual position 36 reached by the distal end of the inner sleeve 34 in accordance with the display magnification, based on predetermined distance information from the outer sleeve 32 to the distal end of the inner sleeve 34 in a case where the inner sleeve 34 protrudes from the outer sleeve 32.

Further, since the virtual position 36 reached by the distal end of the inner sleeve 34 varies depending on the cross section to be displayed, the controller 24 calculates the position of the distal end of the inner sleeve 34 according to the cross section of the CT image 38 to be displayed, based on predetermined distance information from the outer sleeve 32 to the distal end of the inner sleeve 34 in a case where the inner sleeve 34 protrudes from the outer sleeve 32, and superimposes and displays the virtual position 36 on the CT image 38.

For example, the CT image 38 of an axial cross section orthogonal to the biopsy needle 30, a sagittal cross section passing through the central axis of the biopsy needle 30 and orthogonal to the axial cross section, and a coronal cross section orthogonal to the sagittal cross section of the needle tip can be displayed. In addition, in a sagittal cross section, as shown in FIG. 6, it is assumed that the angle of the outer sleeve 32 is inclined by an angle θ1 from the axial (body axis cross section). In this case, in the sagittal cross section, the distance for the superimposition of the inner sleeve 34 is fixed at a predetermined distance. FIG. 6 is a diagram showing an example of the virtual position 36 of the inner sleeve 34 displayed on a sagittal cross section.

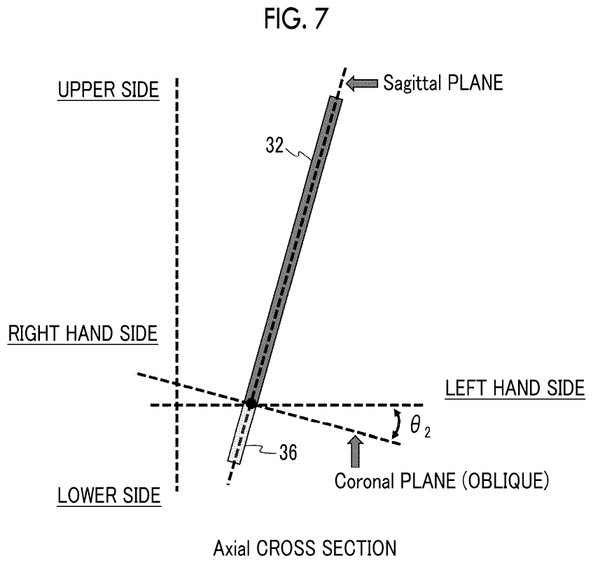

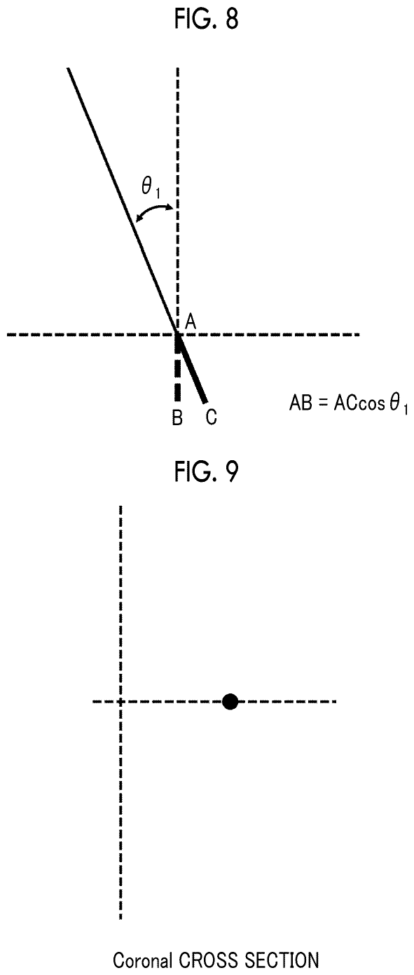

On the other hand, as shown in FIG. 7, in the axial cross section, since the outer sleeve 32 is inclined by the angle θ1 in the sagittal cross section (FIG. 6), the display distance AB is predetermined distance (AC)×cos θ1 at which the inner sleeve 34 protrudes as shown in FIG. 8, in a case of viewing the axial cross section. FIG. 7 is a diagram showing an example of the virtual position 36 of the inner sleeve 34 displayed in the axial cross section, and FIG. 8 is a diagram showing an example of a distance at which the virtual position 36 of the inner sleeve 34 is displayed in the axial cross section.

In addition, in the coronal cross section (oblique), only the point of the needle tip is displayed as shown in FIG. 9. FIG. 9 is a diagram showing an example of the virtual position 36 of the inner sleeve 34 displayed on a coronal cross section.

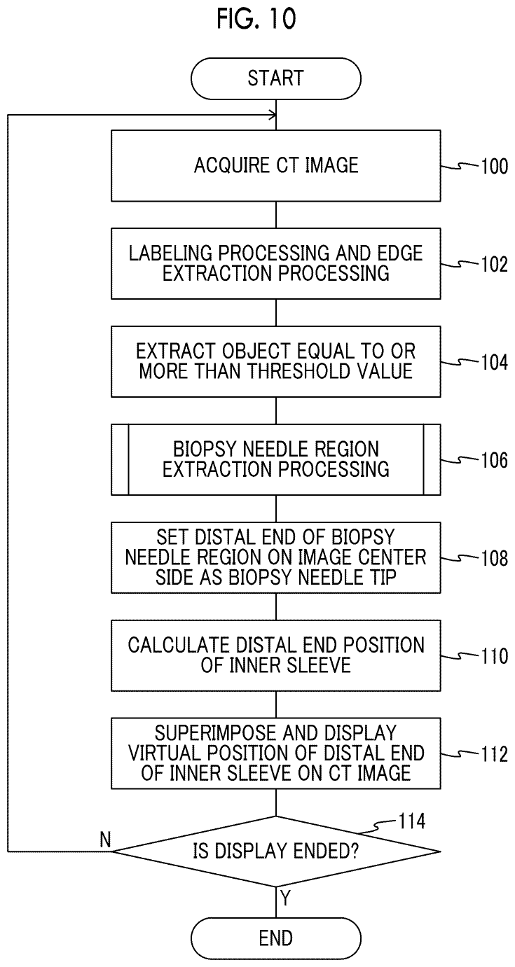

Subsequently, specific processing performed by the controller 24 of the medical image examination apparatus 10 according to the present embodiment configured as described above will be described. FIG. 10 is a flowchart showing an example of a flow of processing performed by the controller 24 of the medical image examination apparatus 10 according to the present embodiment. The processing in FIG. 10 is started, for example, in a case where the operation panel 22 is operated and the imaging of the CT image 38 of the CT-guided biopsy is instructed.

In step 100, the controller 24 acquires the CT image 38 and proceeds to step 102. That is, the CT image 38 acquired by the gantry 12 is acquired.

In step 102, the controller 24 performs the labeling processing and the edge processing on the acquired CT image 38 and proceeds to step 104.

In step 104, the controller 24 extracts the object equal to or greater than the predetermined threshold value and proceeds to step 106. That is, an average density value (CT value) at each pixel in the object labeled by the labeling processing is calculated, and an object within a CT value range equal to or greater than a preset threshold value is extracted.

In step 106, the controller 24 performs the biopsy needle region extraction processing and proceeds to step 108. In the biopsy needle region extraction processing, an object corresponding to at least one of Condition 1 or Condition 2 described above is extracted in order to distinguish the bone having a CT value close to that of the biopsy needle 30 (metal). Here, in a case where an object corresponding to each of Condition 1 and Condition 2 is extracted, in a case where the object corresponding to Condition 1 and the object corresponding to Condition 2 are different from each other, the object corresponding to Condition 2 may be set as the biopsy needle region. In addition, for Condition 1 and Condition 2, the region corresponding to Condition 2 may be extracted, the region corresponding to Condition 2 may be set as the biopsy needle region in a case where the region corresponding to Condition 2 exists, and in a case the region corresponding to Condition 2 does not exist, the region corresponding to Condition 1 may be extracted, and the region corresponding to Condition 1 may be set as the biopsy needle region in a case where the region corresponding to Condition 1 exists.

In step 108, the controller 24 sets the distal end of the biopsy needle region on the image center side as the biopsy needle tip and proceeds to step 110.

In step 110, the controller 24 calculates the distal end position of the inner sleeve 34 and proceeds to step 112. For example, a pixel corresponding to the predetermined protrusion distance of the inner sleeve 34 in the central axis direction of the biopsy needle 30 from the pixel of the biopsy needle tip is calculated as the distal end position. In addition, in a case where the display magnification can be changed, the virtual position 36 reached by the distal end of the inner sleeve 34 in accordance with the display magnification is calculated based on predetermined distance information from the outer sleeve 32 to the distal end of the inner sleeve 34 in a case where the inner sleeve 34 protrudes from the outer sleeve 32. Further, the position of the distal end of the inner sleeve 34 according to the cross section of the displayed CT image 38 is calculated based on the predetermined distance information from the outer sleeve 32 to the distal end of the inner sleeve 34 in a case where the inner sleeve 34 protrudes from the outer sleeve 32.

In step 112, the controller 24 superimposes and displays the virtual position 36 of the distal end of the inner sleeve 34 on the CT image 38, and proceeds to step 114. For example, as shown in FIG. 4, the inner sleeve 34 protruding from the outer sleeve 32 may be displayed with an arrow as a virtual position 36, and the virtual position 36 reached by the distal end of the inner sleeve 34 protruding from the outer sleeve 32 may be displayed as a tip of the arrow. In addition, in a case of displaying the virtual position 36 reached by the distal end of the inner sleeve 34, the virtual position 36 may be superimposed and displayed on the CT image 38 in at least one display aspect of a predetermined color or transparency as the inner sleeve 34 that protrudes by a pixel of a predetermined protrusion distance of the inner sleeve 34 in a central axis direction of the biopsy needle 30 from a pixel of the biopsy needle tip.

In step 114, the controller 24 determines whether an instruction to end the display is issued. In a case where the determination is negative, the process returns to step 100, and the above-described process is repeated. In a case where the determination is affirmative, the series of processes is ended.

By performing the processing by the controller 24 in this way, the virtual position 36 reached by the inner sleeve 34 can be confirmed with the CT image 38 without manually performing the distance measurement of the inner sleeve 34 protruding from the outer sleeve 32. In addition, it is not necessary to manually measure the distance again even in a case where the positional relationship between the distal end 32A of the outer sleeve 32 of the biopsy needle 30 and the target is changed, and the time required for the CT-guided biopsy can be shortened as compared with the related art.

In addition, since the virtual position 36 reached by the inner sleeve 34 is displayed on the CT image 38, it is possible to easily confirm whether the outer sleeve 32 and the target are on the same straight line, and thus, the time required for the CT-guided biopsy can be shortened as compared with the related art.

In the above-described embodiment, the biopsy needle region is extracted by performing the labeling processing, the edge extraction processing, and the like, but the present disclosure is not limited thereto. For example, the user may click once on the region in the biopsy needle on the CT image, fill the pixel of the nearby CT value with image processing, and treat the region as the biopsy needle region. That is, the user may designate the biopsy needle region by clicking the region in the biopsy needle on the CT image once, acquire designation information of the biopsy needle region on the radiation image designated by the user, and extract the biopsy needle region by filling pixels within a density value range equal to or more than a preset threshold value around the designation information based on the designation information.

In addition, in the above-described embodiment, an example of the radiation image from which the CT image 38 is acquired has been described, but the radiation image is not limited to the CT image 38. For example, an X-ray image acquired by an X-ray fluoroscopy stand may be applied, or a computed radiography (CR) image may be applied. Alternatively, an image using an extended reality (XR) technology may be applied.

In addition, in the above-described embodiment, the processing performed by the controller 24 has been described as processing performed by software, but may be realized by a dedicated hardware circuit. In this case, the processing may be executed by one hardware or may be executed by a plurality of hardware.

In addition, in the embodiment described above, the processor refers to a processor in a broad sense, and examples of the processor include a general-purpose processor (for example, a central processing unit (CPU) or the like), and a dedicated processor (for example, a graphics processing unit (GPU), an application specific integrated circuit (ASIC), a field programmable gate array (FPGA), a programmable logic device, or the like).

In addition, the operation of the processor in the embodiment described above may be performed not only by one processor but also by cooperation of a plurality of processors provided at physically separated positions. In addition, the order of the operations of the processor is not limited to only the order described in the embodiments and may be changed as appropriate.

In addition, the program may be provided by a computer-readable non-transitory recording medium such as a universal serial bus (USB) memory, a flexible disk, or a compact disc read only memory (CD-ROM), or may be provided online through a network such as the Internet. In this case, the program recorded on the computer-readable non-transitory recording medium is usually transferred to and stored in a memory, a storage, or the like. In addition, this program may be provided, for example, as a single application software, or may be incorporated into software of each device as one function of the device.

In addition, a program according to the present disclosure can be provided as a program product. The program product includes products in all aspects for providing a program. For example, the program product includes a program provided through a network such as the Internet, and a non-transitory computer readable recording medium such as a CD-ROM or a DVD in which the program is stored.

In addition, the configuration, operation, and the like of the medical image examination apparatus 10 described in the above-described embodiment are merely examples, and it goes without saying that the configuration, operation, and the like can be changed according to the situation without departing from the spirit of the present disclosure.

In regard to the above-described embodiments, the supplementary notes will be further disclosed as follows.

### Supplementary Note 1

An image processing apparatus comprising a processor,
in which the processor is configured to:
acquire a radiation image of a subject including a biopsy needle in which an inner sleeve protrudes from an outer sleeve;
detect a biopsy needle region in the radiation image; and
based on a result of the detection, superimpose and display on the radiation image a virtual position reached by a distal end of the inner sleeve protruding from the outer sleeve.

### Supplementary Note 2

The image processing apparatus according to Supplementary Note 1,
in which the processor is configured to superimpose and display the virtual position on the radiation image based on predetermined distance information to the distal end of the inner sleeve in a case where the inner sleeve protrudes from the outer sleeve.

### Supplementary Note 3

The image processing apparatus according to Supplementary Note 2,
in which the processor is configured to, based on the predetermined distance information, calculate a position of the distal end of the inner sleeve in accordance with a display magnification, and superimpose and display the virtual position on the radiation image.

### Supplementary Note 4

The image processing apparatus according to Supplementary Note 2,
in which the processor is configured to, based on the predetermined distance information, calculate a position of the distal end of the inner sleeve in accordance with a cross section of the radiation image to be displayed, and superimpose and display the virtual position on the radiation image.

### Supplementary Note 5

The image processing apparatus according to any one of Supplementary Notes 1 to 4,
in which the processor is configured to:
perform labeling processing and edge processing on the radiation image;
calculate an average radiation density value for each pixel within an object labeled by the labeling processing; and
detect, among objects with a density value range equal to or more than a preset threshold value, at least one object having a region that extends radially from an image center by a distance equal to or greater than a predetermined distance or an object having a region that extends by a distance equal to or greater than a predetermined distance in a boundary region between air and a body surface and in an air-side region, and set the detected object as a biopsy needle region.

### Supplementary Note 6

The image processing apparatus according to Supplementary Note 5,
in which, among the objects with the density value range equal to or more than the preset threshold value, in a case of detecting the object having the region that extends radially from the image center by the distance equal to or greater than the predetermined distance or the object having the region that extends by the distance equal to or greater than the predetermined distance in the boundary region between the air and the body surface and in the air-side region, the processor is configured to first detect the object having the region that extends by the distance equal to or greater than the predetermined distance in the boundary region between the air and the body surface and in the air-side region, and set the object as a biopsy needle region in a case where the object is detected.

### Supplementary Note 7

The image processing apparatus according to Supplementary Note 6,
in which the processor is configured to detect the object having the region that extends by the distance equal to or greater than the predetermined distance in the boundary region between the air and the body surface and in the air-side region, and in a case where the object is not detected, detect the object having the region that extends radially from the image center by the distance equal to or greater than the predetermined distance.

### Supplementary Note 8

The image processing apparatus according to Supplementary Note 5,
in which the processor is configured to:
calculate a central pixel between edges of the biopsy needle region and set the central pixel as a pixel of a central axis of the biopsy needle; and
superimpose and display the virtual position on the radiation image in a display aspect of at least one of a predetermined color or a predetermined transparency as an inner sleeve that protrudes by a pixel of a protrusion distance of the inner sleeve in a central axis direction of the biopsy needle from a pixel of the distal end of the biopsy needle.

### Supplementary Note 9

The image processing apparatus according to Supplementary Note 8,
in which the processor is configured to calculate the central pixel by interpolating the pixel in a case where edge information is insufficient at the distal end of the biopsy needle.

### Supplementary Note 10

The image processing apparatus according to any one of Supplementary Notes 1 to 4,
in which the processor is configured to:
acquire designation information on a biopsy needle region on the radiation image, which is designated by a user;
fill pixels in a density value range equal to or more than a preset threshold value around the designation information based on the designation information to extract the biopsy needle region; and
set, after the biopsy needle region is extracted, a distal end of a region on a center side of the radiation image as a biopsy needle tip.

### Supplementary Note 11

An image processing method for causing a computer to execute processing, the method comprising:
acquiring a radiation image of a subject including a biopsy needle in which an inner sleeve protrudes from an outer sleeve;
detecting a biopsy needle region in the radiation image; and
based on a result of the detection, superimpose and display a position of a distal end of the inner sleeve on the radiation image as in a case where the inner sleeve protrudes from the outer sleeve, before the inner sleeve protrudes from the outer sleeve.

### Supplementary Note 12

An image processing program for causing a computer to execute processing of:
acquiring a radiation image of a subject including a biopsy needle in which an inner sleeve protrudes from an outer sleeve;
detecting a biopsy needle region in the radiation image; and
based on a result of the detection, superimpose and display a position of a distal end of the inner sleeve on the radiation image as in a case where the inner sleeve protrudes from the outer sleeve, before the inner sleeve protrudes from the outer sleeve.

What is claimed is:

1. An image processing apparatus comprising a processor,
wherein the processor is configured to:
acquire a radiation image of a subject including a biopsy needle in which an inner sleeve protrudes from an outer sleeve;
detect a biopsy needle region in the radiation image; and
based on a result of the detection, superimpose and display on the radiation image a virtual position reached by a distal end of the inner sleeve protruding from the outer sleeve.

2. The image processing apparatus according to claim 1,
wherein the processor is configured to superimpose and display the virtual position on the radiation image based on predetermined distance information to the distal end of the inner sleeve in a case where the inner sleeve protrudes from the outer sleeve.

3. The image processing apparatus according to claim 2,
wherein the processor is configured to, based on the predetermined distance information, calculate a position of the distal end of the inner sleeve in accordance with a display magnification, and superimpose and display the virtual position on the radiation image.

4. The image processing apparatus according to claim 2,
wherein the processor is configured to, based on the predetermined distance information, calculate a position of the distal end of the inner sleeve in accordance with a cross section of the radiation image to be displayed, and superimpose and display the virtual position on the radiation image.

5. The image processing apparatus according to claim 1,
wherein the processor is configured to:
perform labeling processing and edge processing on the radiation image;
calculate an average radiation density value for each pixel within an object labeled by the labeling processing; and
detect, among objects with a density value range equal to or more than a preset threshold value, at least one object having a region that extends radially from an image center by a distance equal to or greater than a predetermined distance, or an object having a region that extends by a distance equal to or greater than a predetermined distance in a boundary region between air and a body surface and in an air-side region, and set the detected object as a biopsy needle region.

6. The image processing apparatus according to claim 5,
wherein, among the objects with the density value range equal to or more than the preset threshold value, in a case of detecting the object having the region that extends radially from the image center by the distance equal to or greater than the predetermined distance or the object having the region that extends by the distance equal to or greater than the predetermined distance in the boundary region between the air and the body surface and in the air-side region, the processor is configured to first detect the object having the region that extends by the distance equal to or greater than the predetermined distance in the boundary region between the air and the body surface and in the air-side region, and set the object as a biopsy needle region in a case where the object is detected.

7. The image processing apparatus according to claim 6,
wherein the processor is configured to detect the object having the region that extends by the distance equal to or greater than the predetermined distance in the boundary region between the air and the body surface and in the air-side region, and in a case where the object is not detected, detect the object having the region that extends radially from the image center by the distance equal to or greater than the predetermined distance.

8. The image processing apparatus according to claim 5,
wherein the processor is configured to:
calculate a central pixel between edges of the biopsy needle region and set the central pixel as a pixel of a central axis of the biopsy needle; and
superimpose and display the virtual position on the radiation image in a display aspect of at least one of a predetermined color or a predetermined transparency as an inner sleeve that protrudes by a pixel of a protrusion distance of the inner sleeve in a central axis direction of the biopsy needle from a pixel of the distal end of the biopsy needle.

9. The image processing apparatus according to claim 8,
wherein the processor is configured to calculate the central pixel by interpolating the pixel in a case where edge information is insufficient at the distal end of the biopsy needle.

10. The image processing apparatus according to claim 1,
wherein the processor is configured to:
acquire designation information on a biopsy needle region on the radiation image, which is designated by a user;
fill pixels in a density value range equal to or more than a preset threshold value around the designation information based on the designation information to extract the biopsy needle region; and
set, after the biopsy needle region is extracted, a distal end of a region on a center side of the radiation image as a biopsy needle tip.

11. An image processing method, the method comprising:
acquiring a radiation image of a subject including a biopsy needle in which an inner sleeve protrudes from an outer sleeve;
detecting a biopsy needle region in the radiation image; and;
based on a result of the detection, superimposing and displaying a position of a distal end of the inner sleeve on the radiation image as in a case where the inner sleeve protrudes from the outer sleeve, before the inner sleeve protrudes from the outer sleeve.

12. A non-transitory computer-readable storage medium storing an image processing program executable by a computer to execute processing of:
acquiring a radiation image of a subject including a biopsy needle in which an inner sleeve protrudes from an outer sleeve;
detecting a biopsy needle region in the radiation image; and
based on a result of the detection, superimposing and displaying a position of a distal end of the inner sleeve on the radiation image as in a case where the inner sleeve protrudes from the outer sleeve, before the inner sleeve protrudes from the outer sleeve.
